(19) United States
(12) Patent Application Publication
(10) Pub. No.: US 2013/0197238 A1
(43) Pub. Date: Aug. 1, 2013

(54) FIPRONIL PRODUCTION PROCESS

(71) Applicants: Anat Levin, Beer Sheva (IL); Michael Grabarnick, Meitar (IL)

(72) Inventors: Anat Levin, Beer Sheva (IL); Michael Grabarnick, Meitar (IL)

(73) Assignee: IRVITA PLANT PROTECTION, A BRANCH OF CELSIUS PROPERTY B.V

(21) Appl. No.: 13/809,327

(22) PCT Filed: Jul. 10, 2011
(86) PCT No.: PCT/IL2011/000546

§ 371 Date: Mar. 14, 2013

Related U.S. Application Data
(60) Provisional application No. 61/363,366, filed on Jul. 12, 2010.

(51) Int. Cl. C07D 231/44

(52) U.S. Cl. CPC C07D 231/44
USPC 548/367.4

(57) ABSTRACT

An improved oxidation process for preparing 5-amino-3-cyano-1-(2,6-dichloro-4-trifluoromethylphenyl)-4-trifluoromethylsulphinyl-pyrazole, of formula (I) is described. The process includes admixing 5-amino-3-cyano-1-(2,6-dichloro-4-trifluoromethylphenyl)-4-trifluoromethylthiopyrazole of formula (II) with dichloroacetic acid and hydrogen peroxide in the presence of a strong acid.

### BACKGROUND OF THE INVENTION

This disclosure relates to a process for the production of fipronil from the corresponding sulfide.

Fipronil, 5-amino-3-cyano-1-(2,6-dichloro-4-trifluoromethylphenyl)-4-trifluoromethylsulphinyl-pyrazole (CAS Registry No. 120068-37-3), is represented by the following structural formula I.

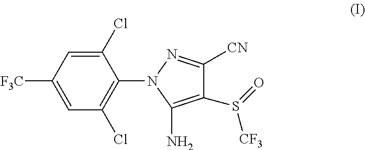 [SMILES: N#Cc1nn(-c2c(Cl)cc(C(F)(F)F)cc2Cl)c(N)c1S(=O)C(F)(F)F]

Fipronil is a highly active, broad-spectrum use insecticide that belongs to the phenylpyrazole chemical family. Fipronil selectively acts by blocking the GABA-gated chloride channels of neurons in the central nervous system and causes neural excitation and convulsions in insects, resulting in death.

Fipronil was discovered and developed by Rhone-Poulenc between 1985 and 1987 and placed on the market in 1993. It was first introduced to the U.S. in 1996 for commercial turf and indoor pest control. It is mostly used to control ants, beetles, cockroaches, fleas, ticks, termites, mole crickets, thrips, rootworms, weevils, and other insects.

Fipronil is used in a wide variety of pesticide products, including granular products for grass, gel baits, spot-on pet care products, liquid termite control products, and products for agriculture.

The synthesis and use of fipronil was described in several patents, for example in European Patent Publication No. 295,117. The final step of the process described therein involves an oxidation reaction carried out by reacting the compound 5-amino-3-cyano-1-(2,6-dichloro-4-trifluoromethylphenyl)-4-trifluoromethylthiopyrazole of formula (II) with m-chloroperbenzoic acid in dichloromethane for more than two days. The residue is purified by means of a silica gel column chromatography to afford fipronil of formula (I) in 58% yield, as depicted in Scheme 1.

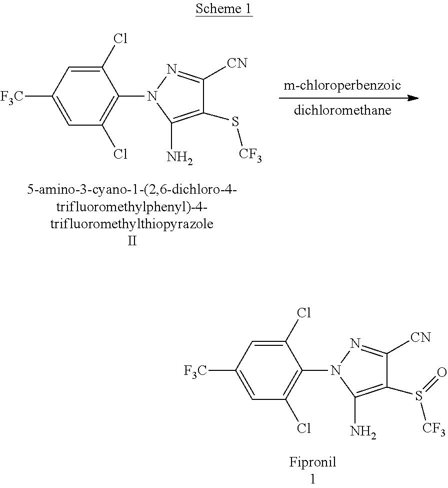 [SMILES: N#Cc1nn(-c2c(Cl)cc(C(F)(F)F)cc2Cl)c(N)c1SC(F)(F)F ; N#Cc1nn(-c2c(Cl)cc(C(F)(F)F)cc2Cl)c(N)c1S(=O)C(F)(F)F]

The process as described in European Patent Publication No. 295,117 has, however, some disadvantages. The oxidizing agent m-chloroperbenzoic acid is a highly explosive and expensive reagent, and is, therefore, not a preferred reagent for use in commercial scale production. Additionally, the process is disadvantageous in that it is lengthy; fipronil is purified by means of a silica gel column chromatography; and fipronil is obtained in a relatively low yield of 58%, which makes this process unattractive for industrial implementation.

European Patent Publication No. 1,222,173 describes another process for preparing fipronil of formula (I) by oxidizing the compound 5-amino-3-cyano-1-(2,6-dichloro-4-trifluoromethylphenyl)-4-trifluoromethylthiopyrazole of formula (II), at a reduced temperature of 12° C., with a combination of hydrogen peroxide and trifluoroacetic acid which generates in situ trifluoroperacetic acid as an oxidant to give fipronil of formula (I) in 89% yield. It is mentioned by the inventors of European Patent Publication No. 1,222,173 that a drawback of using the trifluoroacetic acid and hydrogen peroxide mixture on large scales is that it leads to corrosion of the glass linings of industrial reaction vessels and that the addition of a corrosion inhibiting compound such as boric acid to the reaction mixture inhibits the corrosion process and reduces the speed of corrosion. Though hydrogen peroxide is a low cost reagent, trifluoroacetic acid is relatively expensive chemical which needs to be recovered due to process economics, thereby increasing the cost of this route.

International Patent Application Publication No. WO 2007/122440 (hereinafter the '440 application) describes yet another process for preparing fipronil of formula (I) by oxidizing the compound 5-amino-3-cyano-1-(2,6-dichloro-4-trifluoromethylphenyl)-4-trifluoromethylthiopyrazole of formula (II) in a medium comprising hydrogen peroxide and trichloroacetic acid which forms trichloroperacetic acid in situ as the reactive species. Since trichloroacetic acid is a solid under the conditions of oxidation, at least one melting point depressant, such as methylene dichloride, is required. It is also mentioned by the inventors of the '440 application that mineral acids (i.e., inorganic acids) are generally not useful as a medium for oxidation due to the instability of the compounds of formula (II) or formula (I) towards strong mineral acids. The use of chlorinated hydrocarbon, such as methylene chloride, chloroform, carbontetrachloride and ethylene dichloride, is not particularly desirable for industrial implementation due to the hazards associated with such solvents. Owing to the economy of the process, the relatively expensive trichloroacetic acid should be recovered and recycled after reaction, which is almost impractical because of its high melting point.

Based on the disadvantages in the above processes, it would be highly desirable to have an improved process for the production of fipronil which is suitable for industrial use, simple, low-cost, highly efficient and environmentally friendly, thereby overcoming the deficiencies of the prior art. The present invention provides a process having one or more of the foregoing advantages.

### BRIEF SUMMARY OF THE INVENTION

The present invention provides an improved oxidation process for preparing 5-amino-3-cyano-1-(2,6-dichloro-4-trifluoromethylphenyl)-4-trifluoromethylsulphinyl-pyrazole, fipronil, of formula (I) in high yield, which process overcomes the disadvantages of the known methods for preparing fipronil. The process includes:

admixing 5-amino-3-cyano-1-(2,6-dichloro-4-trifluoromethylphenyl)-4-trifluoromethylthiopyrazole of formula (II), with dichloroacetic acid and hydrogen peroxide in the presence of a strong acid and allowing the oxidation reaction to proceed for a time period sufficient to allow substantial completion of the oxidation reaction, to produce the compound of formula (I) in a reaction mixture;
quenching the reaction mixture;
isolating the compound of formula (I) from the quenched reaction mixture; and
optionally purifying the obtained compound of formula (I).

The compound of formula (I) can be isolated and purified by any suitable method, which can include, for example, precipitation, crystallization, slurrying, washing in a suitable solvent, filtration through a packed-bed column, dissolution in an appropriate solvent and re-precipitation by addition of a second solvent in which the compound is insoluble, or any combination of such purification methods.

### DETAILED DESCRIPTION OF THE INVENTION

The applicants have surprisingly found that 5-amino-3-cyano-1-(2,6-dichloro-4-trifluoromethylphenyl)-4-trifluoromethylthiopyrazole of formula (II) can be oxidized directly with dichloroacetic acid and hydrogen peroxide in the presence of a strong acid.

The process described herein is advantageous in that it avoids the need for using hazardous and expensive oxidizing reagents. The process also avoids the need for using dichloromethane, which is not particularly desirable for industrial implementation due to the hazards associated with such solvent.

Thus the process of the present invention includes:

admixing 5-amino-3-cyano-1-(2,6-dichloro-4-trifluoromethylphenyl)-4-trifluoromethylthiopyrazole of formula (II), with dichloroacetic acid and hydrogen peroxide in the presence of a strong acid and allowing the oxidation reaction to proceed for a time period sufficient to allow substantial completion of the oxidation reaction, to produce the compound of formula (I) in a reaction mixture;
quenching the reaction mixture;
isolating the compound of formula (I) from the quenched reaction mixture; and
optionally purifying the obtained compound of formula (I).

The reaction can be conducted in an organic solvent. Examples of organic solvents that can be used in the present invention include monochlorobenzene, poly chlorobenzene, toluene, xylene, ethyl acetate, butyl acetate, acetonitrile, N-methylpyrrolidone (NMP) and dimethylacetamide (N,N-DMA), or a combination thereof.

Dichloroacetic acid is generally present in molar excess. For example, the molar excess of dichloroacetic acid ranges from about 2 molar equivalents to about 50 molar equivalents, preferably from about 4.5 molar equivalents to about 30 molar equivalents per one mol of the 5-amino-3-cyano-1-(2,6-dichloro-4-trifluoromethylphenyl)-4-trifluoromethylthiopyrazole of formula (II). Dichloroacetic acid can be used, together with the strong acid, as the solvent for the reaction mixture.

Suitable strong acids include sulfuric acid, methanesulfonic acid and p-toluenesulfonic acid, or a combination thereof. The strong acid is generally present in an amount effective to catalyze the oxidation. For example, the molar ratio of the strong acid to the 5-amino-3-cyano-1-(2,6-dichloro-4-trifluoromethylphenyl)-4-trifluoromethylthiopyrazole of formula (II) is from 1:1 to 5:1.

In an embodiment, the oxidizing agent utilized in the process disclosed herein, perdichloroacetic acid (PAA) is optionally formed in situ from dichloroacetic acid and hydrogen peroxide.

According to the present invention, when the oxidizing agent is prepared in situ hydrogen peroxide is added gradually over time. For example, the hydrogen peroxide is added drop-wise to the mixture of 5-amino-3-cyano-1-(2,6-dichloro-4-trifluoromethylphenyl)-4-trifluoromethylthiopyrazole of formula (II), dichloroacetic acid and strong acid over a period of from 30 minutes to about 120 minutes, more specifically, over a period of from 50 minutes to about 100 minutes, more specifically over a period of from 65 minutes to about 90 minutes.

In another embodiment, the oxidizing agent utilized in the process disclosed herein, perdichloroacetic acid (PAA) is added to the reaction mixture gradually over time. For example, the oxidizing agent is added drop-wise to the solution of 5-amino-3-cyano-1-(2,6-dichloro-4-trifluoromethylphenyl)-4-trifluoromethylthiopyrazole of formula (II) dissolved in organic solvent over a period of from 30 minutes to about 240 minutes, more specifically, over a period of from 90 minutes to about 180 minutes.

Hydrogen peroxide is used in the form of aqueous solutions, for example in the form of the usual commercial-available solutions, which have a concentration ranging from 30 to 70% by weight.

In an embodiment, the process is conducted at a temperature in the range of from about 0° C. to about 40° C., more specifically from about 5° C. to about 15° C.

The progress of the reaction can be monitored using any suitable method, which can include, for example, chromatographic methods such as, e.g., high performance liquid chromatography (HPLC), thin layer chromatography (TLC), and the like. The reaction may be quenched after nearly complete disappearance of the starting material 5-amino-3-cyano-1-(2,6-dichloro-4-trifluoromethylphenyl)-4-trifluoromethylthiopyrazole of formula (II) as determined by one or more of such methods.

The oxidation process can be quenched by mixing the reaction mixture with a suitable quenching agent. Examples of quenching agents include sodium metabisulfite, sodium sulfite, sodium thiosulfate and buffers such as phosphate buffer (NaH₂PO₄/Na₂HPO₄), carbonate buffer (NaHCO₃/NaCO₃) and acetate buffer (CH₃CO₂H/CH₃CO₂Na), or a combination thereof.

The use of hydrogen peroxide reduces the cost of production, simplifies work-up and minimizes the effluent disposal problem. This forms another embodiment of the present invention.

In yet another embodiment, the compound of formula (I) can be isolated from the reaction mixture by any conventional techniques well-known in the art selected, without limitation, from the group consisting of concentration, extraction, precipitation, cooling, filtration, crystallization or centrifugation or a combination thereof followed by drying.

In yet another embodiment, the compound of formula (I) can be optionally purified by any conventional techniques well-known in the art selected, without limitation, from the group consisting of precipitation, crystallization, slurrying, washing in a suitable solvent, filtration through a packed-bed column, dissolution in an appropriate solvent and re-precipitation by addition of a second solvent in which the compound is insoluble or any suitable combination of such methods.

The fipronil produced in accordance with process disclosed herein has a purity of greater than about 95%, a purity of greater than about 96%, and more preferably a purity of greater than about 97%. Purity can be determined by HPLC, for example, or other methods known in the art.

The yield of the process is an important feature of the invention. As described in the examples, fipronil can be obtained in a yield of over 95%, more preferably over 96%, more preferably over 97%, with respect to the starting amount of the molecule having the structure formula (II).

The following examples illustrate the practice of the present invention in some of its embodiments, but should not be construed as limiting the scope of the invention. Other embodiments will be apparent to one skilled in the art from consideration of the specification and examples. It is intended that the specification, including the examples, is considered exemplary only without limiting the scope and spirit of the invention.

### Example 1

This example demonstrates the preparation of fipronil. 100 grams (0.23 mol) of 5-amino-3-cyano-1-(2,6-dichloro-4-trifluoromethylphenyl)-4-trifluoromethylthiopyrazole (compound of formula (II)) were dissolved in a mixture consisting of 900 grams (6.97 mol) of dichloroacetic acid (DCAA) and 30 grams (0.3 mol) of H₂SO₄. After 30 minutes of stirring at a temperature of 15° C., 25 grams (0.22 mol) of a 30% w/w aqueous hydrogen peroxide solution were added over a period of 90 minutes. The reaction was continued until the conversion was more than 95% as measured by HPLC. The mixture was quenched by using Na₂SO₃. Isolation and further purification of fipronil was done by the conventional methods. Fipronil was obtained in 98% yield, having a purity of 97.5% (by HPLC).

### EXAMPLES 2-4

The % conversion obtained by reacting the compound of formula (II) with different amounts of acid and of hydrogen peroxide at different reaction temperatures is summarized in Table 1:

TABLE 1

| | | | Reaction | Hydrogen | Hydrogen | |
|---|---|---|---|---|---|---|
| Expt. | | Acid | Temperature | peroxide | peroxide | Con- |
| No. | Acid | gr | ° C. | gr | % | version % |
| 2 | H₂SO₄ | 35 | 30 | 20 | 50 | 97 |
| 3 | H₂SO₄ | 35 | 20 | 20 | 50 | 96 |
| 4 | H₂SO₄ | 76 | 0 | 35 | 30 | 97 |

### Example 5

This example demonstrates the preparation of PAA (Perdichloroacetic Acid). 1250 grams (9.68 mol) of dichloroacetic acid (DCAA) and 400 grams (4 mol) of H₂SO₄ mixed at 5° C. 200 gr (2.05 mol) of a 35% w/w aqueous hydrogen peroxide solution were added over a period of 30 minutes and the mixture was stirred for additional 30 minutes. The solution was used without further purification.

### Example 6

This example demonstrates the preparation of fipronil. 850 grams (2 mol) of 5-amino-1-(2,6-dichloro-4-trifluoromethylphenyl)-3-cyano-4-trifluoromethylthiopyrazole were dissolved in monochlorobenzene at 10° C. A solution of PAA, prepared according to example 5 was added over a period of 180 minutes. At the end of the addition the reaction was quenched by admixing the mixture with a phosphate (NaH₂PO₄/Na₂HPO₄) buffer solution while maintaining the pH neutral followed by the addition of 20% sodium metabisulfite solution. Subsequently, fipronil was isolated and further purified by conventional methods with a molar yield of 98% and purity of 97.5% (by HPLC).

What is claimed is:

1. A process for preparing 5-amino-3-cyano-1-(2,6-dichloro-4-trifluoromethylphenyl)-4-trifluoromethylsulphinyl-pyrazole, fipronil, of formula (I) 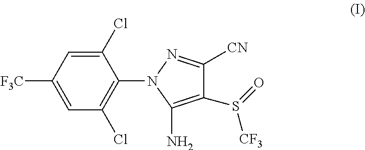 [SMILES: N#Cc1nn(-c2c(Cl)cc(C(F)(F)F)cc2Cl)c(N)c1S(=O)C(F)(F)F]
wherein the process comprises:
admixing 5-amino-3-cyano-1-(2,6-dichloro-4-trifluoromethylphenyl)-4-trifluoromethylthiopyrazole of formula (II) 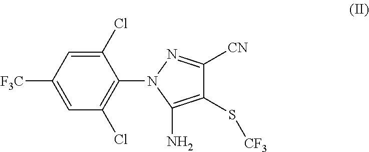 [SMILES: N#Cc1nn(-c2c(Cl)cc(C(F)(F)F)cc2Cl)c(N)c1SC(F)(F)F]
with dichloroacetic acid and hydrogen peroxide in the presence of a strong acid;
quenching the reaction mixture;
isolating the compound of formula (I); and optionally purifying the obtained compound of formula (I).

2. The process according to claim 1 which further comprises organic solvent selected from monochlorobenzene, polychlorobenzene, toluene, xylene, ethyl acetate, butyl acetate, acetonitrile, N-methylpyrrolidone (NMP) and dimethylacetamide (N,N-DMA), or a combination thereof.

3. The process according to claim 1, wherein said strong acid is selected from sulfuric acid, methanesulfonic acid, and p-toluenesulfonic acid, or a combination thereof.

4. The process according to claim 1, wherein the process is conducted at a temperature in the range of from about 0° C. to about 40° C.

5. The process according to claim 1, which comprises in situ preparation of the oxidizing agent.

6. The process according to claim 4, wherein the hydrogen peroxide is added to the reaction mixture over a period of from 30 minutes to about 120 minutes.

7. The process according to claim 1, wherein the oxidizing agent is added to the reaction mixture over a period of from 30 minutes to about 240 minutes.

8. The process of claim 1, wherein the reaction mixture is quenched by adding a quenching agent selected from the group consisting of sodium metabisulfite, sodium sulfite, sodium thiosulfate and buffers such as phosphate buffer (NaH₂PO₄/Na₂HPO₄), carbonate buffer (NaHCO₃/NaCO₃) and acetate buffer (CH₃CO₂H/CH₃CO₂Na), or a combination thereof.

9. The process according to claim 1, wherein the compound of formula (I) has a purity of at least 95%.
